(19) United States
(12) Patent Application Publication
(10) Pub. No.: US 2015/0160177 A1
(43) Pub. Date: Jun. 11, 2015

(54) METHOD OF MEASURING A PHYSICAL PROPERTY OF A SOLID BODY

(71) Applicant: CONOCOPHILLIPS COMPANY, Houston, TX (US)

(72) Inventors: Dominique P. BERTA, Flower Mound, TX (US); Matthew L. CLINGERMAN, Dubai (AE)

(21) Appl. No.: 14/556,551

(22) Filed: Dec. 1, 2014

Related U.S. Application Data
(60) Provisional application No. 61/912,821, filed on Dec. 6, 2013.

(51) Int. Cl. G01N 33/18; G06K 7/10; G01L 19/00

(52) U.S. Cl. CPC G01N 33/18; G01L 19/00; G06K 7/10405; G01N 2033/1873

(57) ABSTRACT

A method of measuring a physical property of a solid body is provided. The method includes remotely deploying a measurement device into a body of ice prone water. The method also includes embedding the measurement device within an ice pack newly formed within the body of ice prone water. The method further includes storing data of the physical property within the measurement device.

### CROSS-REFERENCE TO RELATED APPLICATIONS

This application is a non-provisional application which claims the benefit of and priority to U.S. Provisional Application Ser. No. 61/912,821 filed Dec. 6, 2013, entitled “METHOD OF MEASURING A PHYSICAL PROPERTY OF A SOLID BODY,” which is hereby incorporated by reference in its entirety.

### FIELD OF THE INVENTION

This invention relates to a method of measuring and remotely collecting data of one or more physical properties of a solid body, such as an ice pack located in ice prone waters.

### BACKGROUND OF THE INVENTION

The accurate measurement of pressure forces within a large material mass is important in a variety of applications. Due to the rapid increase in exploration for and production of oil, gas and other minerals in arctic offshore regions, the measurement of pressure within an arctic ice pack is of particular importance. Certain features, such as ridges, within an arctic ice pack are created by pressure exerted from one floe onto another. The exact distribution of pressure within the ice pack is not fully understood. Accurate prediction of such pressures is important in determining environmental design criteria for arctic offshore and coastal structures. Additionally, continuous monitoring of such pressures is required for the proper defense of such structures. Higher ice pressure increases the structural design requirements, as well as the demand on ice breaking vessels protecting those structures.

Typically, ice pressure must be determined in situ through on-ice istallation of sensing equipment, including strain gauges and accelerometers, for example. Samples removed from the ice pack for subsequent laboratory testing are of limited value since the environmental restraints, once removed, are difficult if not impossible to recreate accurately in a laboratory. Measuring pressures in situ reflects what the ice pack is actually experiencing in terms of pressure, however, prior efforts require on-ice work and the data can be limited due to the scale and complexity required for such on-ice work. In general, the in situ efforts previously employed lead to insufficient and difficult to obtain ice pressure data.

### SUMMARY OF THE INVENTION

In one embodiment of the invention, a method of measuring a physical property of a solid body is provided. The method includes remotely deploying a measurement device into a body of ice prone water. The method also includes embedding the measurement device within an ice pack newly formed within the body of ice prone water. The method further includes storing data of the physical property within the measurement device.

In another embodiment of the invention, a method of measuring a pressure within an ice pack is provided. The method includes remotely deploying a measurement device into a body of ice prone water, wherein the measurement device comprises a strain gauge and a radio frequency identification (RFID) chip. The method also includes embedding the measurement device within the ice pack newly formed upon freezing of the body of ice prone water. The method further includes measuring the pressure within the ice pack with the stain gauge. The method yet further includes storing pressure data measured by the strain gauge on the RFID chip.

### BRIEF DESCRIPTION OF THE DRAWINGS

The invention, together with further advantages thereof, may best be understood by reference to the following description taken in conjunction with the accompanying figures by way of example and not by way of limitation, in which:

FIG. 1 illustrates deployment of measurement devices into a body of ice prone water;

FIG. 2 illustrates the measurement devices embedded within ice packs formed in the body of ice prone water;

FIG. 3 illustrates communication of data measured and stored on the measurement devices to a remote location with a signal;

FIG. 4 is a sample pressure distribution profile analysis based on the data measured by the measurement device; and

FIG. 5 is a flow diagram illustrating a method of measuring a physical property of a solid body.

### DETAILED DESCRIPTION OF THE INVENTION

Reference will now be made in detail to embodiments of the invention, one or more examples of which are illustrated in the accompanying drawings. Each example is provided by way of explanation of the invention, not as a limitation of the invention. It will be apparent to those skilled in the art that various modifications and variation can be made without departing from the scope or spirit of the invention. For instance, features illustrated or described as part of one embodiment can be used on another embodiment to yield a still further embodiment. Thus, it is intended that the invention cover such modifications and variations that come within the scope of the appended claims and their equivalents.

As will be understood from the description below, a method of measuring a physical property of a solid body is provided. In an exemplary embodiment, the solid body is a body of ice or a two-phase ice-water variant. Numerous advantages may be derived from knowledge regarding one or more physical properties of the solid body. For example, accurate data for pressure, temperature and/or density, as well as how those characteristics are distributed throughout the solid body, may provide useful information relating to design criteria for structures or assemblies used in or on the solid body. As noted above, the solid body may be a body of ice. In such an embodiment, the design of offshore and coastal structures located in or near ice prone bodies of water will benefit from the data obtained. In particular, the structures may be suited for the exploration, extraction, and/or production of hydrocarbons.

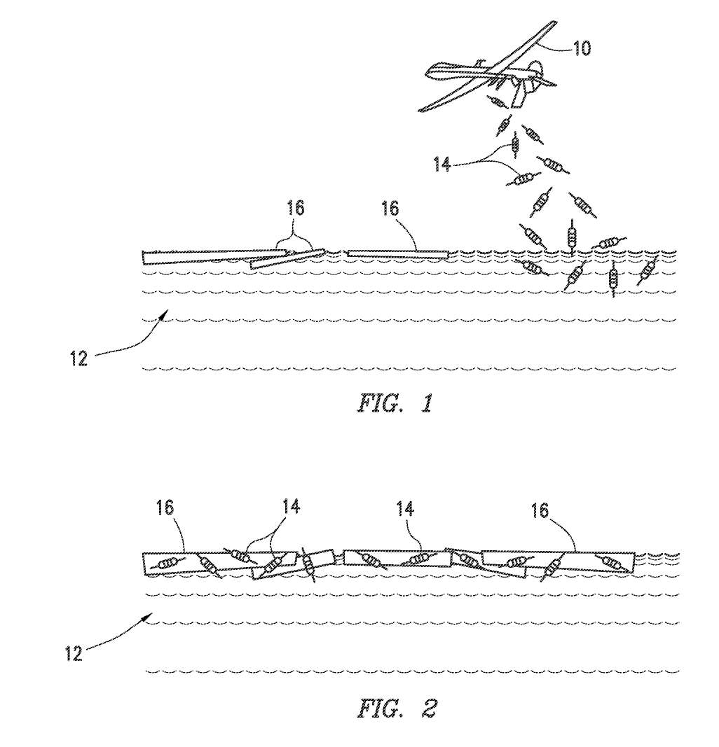

Referring to FIG. 1, an aircraft 10 is shown flying over a body of water 12 that is prone to ice formation near the surface of the body of water 12. The aircraft 10 may be a manned aircraft or an unmanned drone that is remotely operated by a user. The aircraft 10 is loaded with at least one, but typically a plurality of measurement devices, each referenced with numeral 14. The measurement device 14 is remotely deployed by the aircraft 10 into the body of water 12 and is configured to float near the surface of the body of water 12. As portions of the water begin to freeze into one or more ice packs 16, the measurement device 14 is frozen into the ice packs 16. As one can appreciate, the measurement device 14 may become partially or fully embedded within the ice packs 16 as the bodies of ice form. As noted above, the aircraft 10 typically is loaded with a plurality of measurement devices, such that numerous measurement devices become partially or fully embedded within the ice packs 16 upon formation, as shown in FIG. 2, thereby providing a number of devices configured to obtain data in the drop region, as will be appreciated from the description below.

Although the aircraft 10 is illustrated and described as being the vessel configured to remotely deploy the measurement devices 14, it is to be appreciated that other methods of remote distribution are contemplated. For example, a water vessel or a rocket of any type, or any other carrier capable of moving and distributing the measurement devices 14 may be employed to store and distribute the measurement devices.

The measurement device 14 includes a device configured to measure a physical property of the ice pack 16. The physical property to be measured may be any physical property, such as pressure, temperature, density, etc. In an embodiment of the measurement device 14 configured to measure pressure, a strain gauge is included and equipped with a radio frequency identification (RFID) chip. Any conventional strain gauge that fundamentally converts mechanical motion into an electronic signal may be suitable for the embodiments herein, provided the strain gauge is able to withstand the environmental conditions present in ice prone bodies of water. Additionally, the deformation can be measured by mechanical, optical, acoustical, pneumatic, and/or electrical means.

Irrespective of the precise type of strain gauge employed as part of the measurement device 14, the associated RFID chip is configured to store information obtained from the strain gauge. The RFID chip can be passive, active or battery-assisted passive. The RFID chip contains an integrated circuit for storing and processing information, modulating and demodulating a radio frequency (RF) signal and an antenna for receiving and communicating the signal.

The data storage process described above and related to the physical property, such as pressure, is conducted over a period of time to allow the ice packs 16 to naturally deform, move and collide with other ice bodies. The time period of interest is one that is long enough to obtain a detailed picture of the physical property. In one embodiment, the time period is greater than one week. In another embodiment, the time period is greater than one month. In yet another embodiment, the time period of interest is an entire winter. In the case of the physical property being pressure, such exemplary time frames provide a more complete picture of the pressure distribution within the ice packs 16 and in the ice bodies present in the body of water 12.

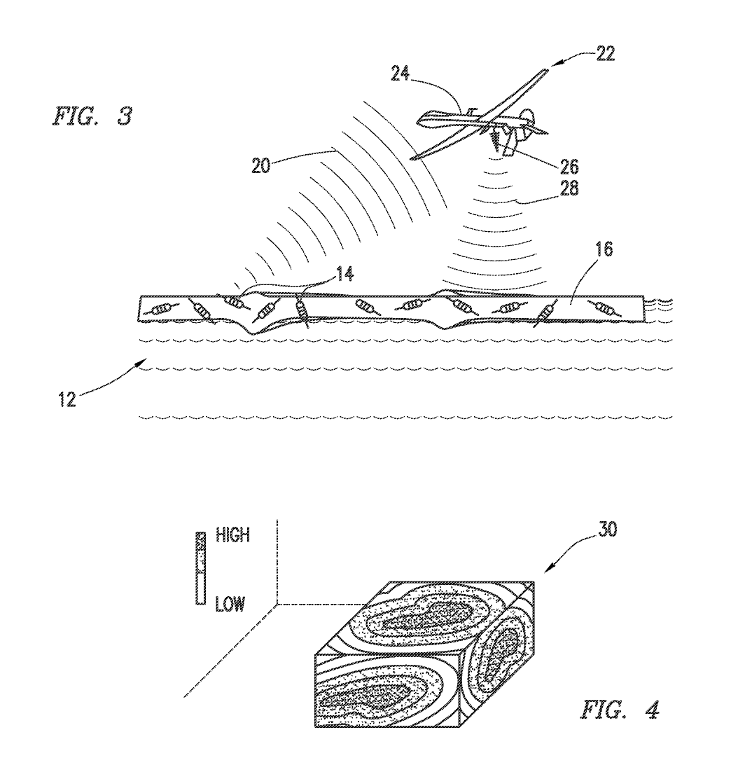

Referring to FIG. 3, the data stored within the measurement device 14 is configured to be communicated via an electronic signal 20 to a remote location 22 via the RFID chip. The RFID chip is in wireless communication with a corresponding RFID reader remotely located. In the illustrated embodiment, the remote location 22 that the electronic signal 20 is communicated to is an aircraft 24, but it is to be understood that alternative embodiments of the remote location 22 may be employed. For example, a water vessel or a land-based structure that is close enough in proximity to the measurement device 14 may be the remote location 22 that the electronic signal 20 is communicated to. In the embodiment of the remote location 22 being the aircraft 24, a manned or unmanned aircraft may be used to fly within range of the electronic signal 20, such as the aircraft 10 used to remotely deploy the measurement devices 14 at the outset of the process. Regardless of the precise structure of the remote location 22, a reader communicates an encoded radio signal 28 to interrogate the RFID chip via an antenna 26 of the remote location 22. The RFID chip receives the message and then responds with the stored data that has been collected over time by the measurement device 14. In certain embodiments, specific tag identification may be communicated as well. Upon communication of the data to the reader, the data is collected onto a device associated with the remote location 22, such as the aircraft 24, which is configured to store and possibly further analyze the data.

The time interval between deployment and collection of data varies depending upon particular application(s) that the data is to be used for. In an ice body or ice-water embodiment, a growing and varying deformation is present due to the changing environmental conditions, as well as movement of, and collision between, the ice packs 16. Therefore, relatively extended periods of time on the order of weeks or months is of interest. As such, communication of the data to the remote location 22 is conducted over a time period ranging from one week to several months (e.g., entire winter). In the illustrated embodiment, the aircraft 24 is flown over the ice packs 16, and thereby the measurement devices 14, periodically to collect the stored data. In particular, the aircraft 24 is flown within range of the electronic signal 20 generated by the RFID chip.

The method described above advantageously provides detailed and accurate data, while eliminating the need to perform any on-ice operations that require the physical presence of humans on the ice packs 16. Such work may be inefficient, costly and potentially dangerous for such workers. Furthermore, data over a larger area can be obtained more efficiently by remotely deploying the measurement devices 14 from the remote location 22, such as the aircraft 10 into freezing water.

Referring to FIG. 4, a sample pressure profile 30 is illustrated. The sample pressure profile 30 is generated as a result of analysis of the data collected and stored by the measurement device 14 that is remotely deployed into the body of water 12. The sample pressure profile 30 provides insight into pressure distributions within the ice packs 16 at particular times of interest. Additionally, the pressure distributions with the ice packs 16 may be analyzed as a function of time.

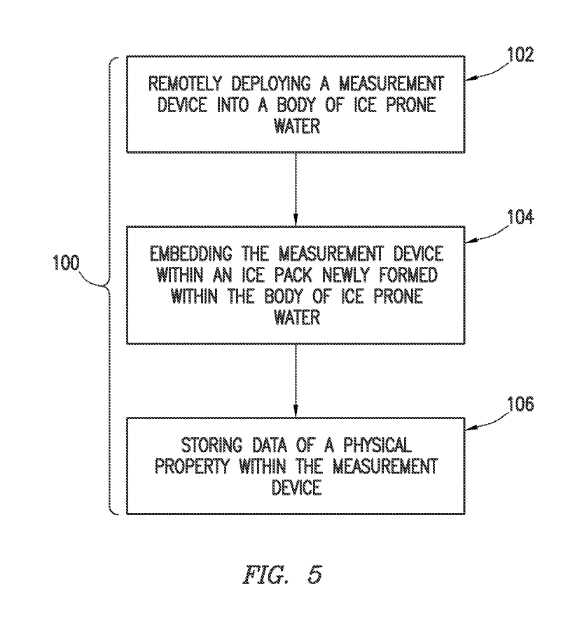

Referring now to FIG. 5, a method of measuring a physical property of a solid body 100 is generally illustrated according to a certain embodiment, with continued reference to FIGS. 1-4. The structural features associated with the method 100 have been previously described and specific structural components need not be described in further detail. The method 100 includes remotely deploying a measurement device into a body of ice prone water 102. The measurement device is at least partially embedded within an ice pack newly formed within the body of ice prone water 104. A physical property is stored 106 within the measurement device and communicated to a remote location for collection and analysis. The embodiment of the method 100 is shown for illustrative purposes and it is to be understood that the preceding description includes several variations of this particular embodiment.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method of measuring a physical property of a solid body, the method comprising:
remotely deploying a measurement device into a body of ice prone water;
embedding the measurement device within an ice pack newly formed within the body of ice prone water; and
storing data of the physical property within the measurement device.

2. The method of claim 1, wherein remotely deploying the measurement device into the body of ice prone water comprises dropping the measurement device from an aircraft.

3. The method of claim 1, wherein the physical property measured comprises pressure within the ice pack.

4. The method of claim 3, wherein remotely deploying the measurement device comprises deploying a strain gauge configured to measure pressure within the ice pack.

5. The method of claim 1, wherein the physical property measured comprises one of temperature and density within the ice pack.

6. The method of claim 1, further comprising communicating the data stored within the measurement device to a remote location.

7. The method of claim 6, wherein the measurement device comprises a radio frequency identification (RFID) chip for processing and storing data.

8. The method of claim 7, wherein the RFID chip communicates the stored data to the remote location.

9. The method of claim 8, the remote location comprising an aircraft, wherein communicating the data stored within the measurement device comprises:
flying the aircraft over the ice pack within a range of a signal communicated by the RFID chip; and
collecting data onto a device located on the aircraft.

10. The method of claim 9, wherein flying the aircraft over the ice pack comprises flying an unmanned drone over the ice pack for collecting data from the RFID chip.

11. The method of claim 9, further comprising flying the aircraft within the range of the signal communicated by the RFID chip over specified time intervals to obtain data over a period of time greater than one week.

12. The method of claim 1, further comprising measuring a pressure distribution within the ice pack over a period of time.

13. The method of claim 12, wherein the period of time is greater than one week.

14. The method of claim 12, wherein the period of time is greater than one month.

15. A method of measuring a pressure within an ice pack, the method comprising:
remotely deploying a measurement device into a body of ice prone water, wherein the measurement device comprises a strain gauge and a radio frequency identification (RFID) chip;
embedding the measurement device within the ice pack newly formed upon freezing of the body of ice prone water;
measuring the pressure within the ice pack with the strain gauge; and
storing pressure data measured by the strain gauge on the RFID chip.

16. The method of claim 15, wherein remotely deploying the measurement device comprises dropping the measurement device from an aircraft.

17. The method of claim 15, further comprising communicating the pressure data from the RFID chip to an aircraft flying within a range of a signal communicated by the RFID chip.

18. The method of claim 17, wherein the aircraft is an unmanned drone.

19. The method of claim 15, further comprising measuring the pressure within the ice pack over a period of time greater than one week.

20. The method of claim 15, further comprising measuring the pressure within the ice pack over a period of time greater than one month.
